(19) United States
(12) Patent Application Publication
(10) Pub. No.: US 2026/0283950 A1
(43) Pub. Date: Sep. 24, 2026

(54) TRI-AZOLE ANTIFUNGAL COMPOSITIONS

(71) Applicant: DERMALIQ THERAPEUTICS INC., Wilmington, DE (US)

(72) Inventors: Frank LÖSCHER, Schriesheim (DE); Markus BEIER, Weinheim (DE); Diana STREHL, Heidelberg (DE)

(21) Appl. No.: 19/472,605

(22) PCT Filed: Mar. 28, 2024
(86) PCT No.: PCT/EP2024/058680

§ 371 Date: Oct. 3, 2025

(30) Foreign Application Priority Data
Apr. 3, 2023 (EP) 23166318.8
Aug. 24, 2023 (EP) 23193141.1

(51) Int. Cl. A61K 9/08; A61K 9/00; A61K 31/454; A61K 47/06; A61K 47/10; A61K 47/14; A61K 47/18; A61K 47/22; A61P 31/10

(52) U.S. Cl. CPC A61K 9/08; A61K 9/0014; A61K 31/454; A61K 47/06; A61K 47/10; A61K 47/14; A61K 47/18; A61K 47/22; A61P 31/10

(57) ABSTRACT

The present invention relates to a liquid composition comprising an azole antifungal agent and a vehicle comprising a semifluorinated alkane.

### BACKGROUND OF THE INVENTION

The present invention is in the field of pharmacotherapy. More specifically, it relates to the treatment of diseases and conditions affecting the nails of humans and other mammals.

While many cutaneous disorders affect the skin itself, there also exist diseases and conditions which relate to the skin appendages, in particular to the nails. These are often difficult to treat due to the thick and dense nail plate largely composed of keratin and its poor uptake of therapeutic agents.

Nails are hardenings of the horny zone of the epidermis. They appear as sheet-like appendages covering the skin on the dorsal side of the terminal phalanges of fingers and toes. The horny zone of the nail is composed of hard alpha-keratin and has a distal, exposed part, or body, and a proximal, hidden portion, or root. The root is covered by a distal prolongation of the stratum corneum of the skin. This narrow fold is composed of soft-keratin and is termed the eponychium. Distal to the eponychium is the “half-moon,” or lunula, a part of the horny zone that is opaque to the underlying capillaries.

Deep to the distal or free border of the nail, the horny zone of the fingertip is thickened and is frequently termed the hyponychium. The horny zone of the nail is attached to the underlying nail bed. The matrix, or proximal part of the bed, produces hard keratin. Further distally, however, the bed may also generate nail substance. Moreover, the most superficial layer of the nail may be produced by the epithelium immediately dorsal to the root and proximal to the eponychium. The growth of the nail is affected by nutrition, hormones, and disease. Nail growth involves considerable protein synthesis, as a result of which nonspecific changes occur in the nails in response to various local and systemic disturbances.

Nails develop in the foetus as epidermal thickenings that undercut the skin to form folds from which the horny substance of the nail grows distally. In adult humans, it takes about 6 months for a fingernail to form, which corresponds to a growth rate of approx. 2-3 mm per month. Toenails tend to grow more slowly than fingernails.

Nails are about two magnitudes thicker than the stratum corneum of the skin. Hard alpha-keratin, the major constituent of the horny zone, is a fibrous structural protein characterised by a high content of cysteine which readily forms thermostable crosslinks via sulphide bridges. The water content of nails is rather low, and typically ranges from about 10 to 30%. Generally speaking, nails are very resistant to the permeation of molecules such as drug substances.

The main function of nails is to protect the sensitive tips of fingers and toes. In addition, fingernails serve in scratching. Toenails are also important for balance.

One of the most common diseases affecting the nail apparatus is fungal infection, also referred to as onychomycosis, a condition affecting about 20% of the adult population in the USA. The incidence is increasing worldwide. In fact, about 30% of all superficial fungal infections affect the nail. Infection may be due to dermatophyte (ringworm, tinea unguium), yeast, or other non-dermatophyte (mould) species. In paronychia, chronic infection of the nail fold is most often caused by Candida species, but bacterial infection with Gram negative species such as Pseudomonas may coexist. Acute paronychia (whitlow) due to staphylococcal infection may also occur, and the presence of these bacterial infections will influence management. Invasion of the nail plate by Candida species may occur in the presence of paronychia, immune deficiency states (including chronic mucocutaneous candidiasis), Raynaud's disease, or endocrine disorders.

Interestingly, conditions of the nails are rather often treated systemically, which itself indicates how difficult it is to achieve a therapeutic drug concentration in the nails by local administration. For example, onychomycosis is treated with oral terbinafine 250 mg daily for 3-6 months, or itraconazole 200 mg daily for 3-6 months. Even the rather conventional therapy with oral griseofulvin 10 mg/kg/daily (500 mg twice daily) for 6-18 months is still being recommended today. In particular proximal nail disease or severe nail bed involvement are being considered as indications for systemic rather than local treatment.

Some topical preparations of known antifungal agents for treatment of onychomycosis exist, such as nail lacquers comprising ciclopirox, amorolfine, or butenafine. While there is some evidence of efficacy, it is believed that successful topical antifungal therapy requires treatment over very long periods, such as a year or even more. Some experts recommend the combination of topical and systemic treatment for better efficacy. Without adequate treatment and patient compliance, the infection will not disappear.

There clearly remains a need for pharmaceutical formulations and vehicles which allow the effective treatment of fungal infections of the nails by topical administration. It is therefore an object of the present invention to provide such improved compositions which overcome one or more disadvantages of known compositions.

### SUMMARY OF THE INVENTION

The invention relates to novel antifungal compositions for use in the treatment of fungal skin infection (mycosis), in particular fungal infections of the nails (onychomycosis), such as finger and toenails of humans and other mammals.

In a first aspect, the invention relates to a liquid composition comprising an azole antifungal agent and a vehicle comprising a semifluorinated alkane.

In a further aspect, the present invention relates to liquid composition comprising an azole antifungal agent and a vehicle comprising a semifluorinated alkane for use as a medicine, in particular for a medicine for treatment of fungal skin infection (mycosis) or fungal infections of the nails (onychomycosis).

In yet a further aspect, the invention prelates to a kit comprising a liquid composition comprising an azole antifungal agent and a vehicle comprising a semifluorinated alkane, and a container for holding the composition, wherein the container is adapted for topical administration of the composition to the skin, in particular to the nail, the folds, the bed, the hyponychium and/or the undersurface of the plate of a toenail and/or a fingernail of a subject.

### DETAILED DESCRIPTION OF THE INVENTION

The present invention relates to novel pharmaceutical compositions for use as topical formulations which are to be administered to the skin, in particular to the nails, preferably to finger and/or toenails of humans and other mammals. The compositions comprise an effective amount of an azole antifungal agent and a liquid vehicle comprising a semifluorinated alkane.

Accordingly, in a first aspect, the invention relates to a liquid composition comprising an azole antifungal agent and a vehicle comprising a semifluorinated alkane. In a particular embodiment of the invention, the liquid composition is a composition comprising an azole antifungal agent and a semifluorinated alkane. Preferably, the liquid composition is a liquid pharmaceutical composition.

As used herein, a pharmaceutical composition is any composition comprising an active ingredient useful for the diagnosis, prevention, management or therapy of a disease, symptom or health condition and at least one carrier or excipient. A topical formulation is a composition which is in a form suitable for topical administration. In the present invention, the composition is suitable for administration to the skin or to the nails of a subject, which subject may be a human or an animal. The nails of the subject, such as finger- or toenails, may be intact or injured, bruised, damaged or otherwise affected.

In preferred embodiments the composition comprises a tri-azole antifungal agent. Suitable tri-azole antifungal agents include:

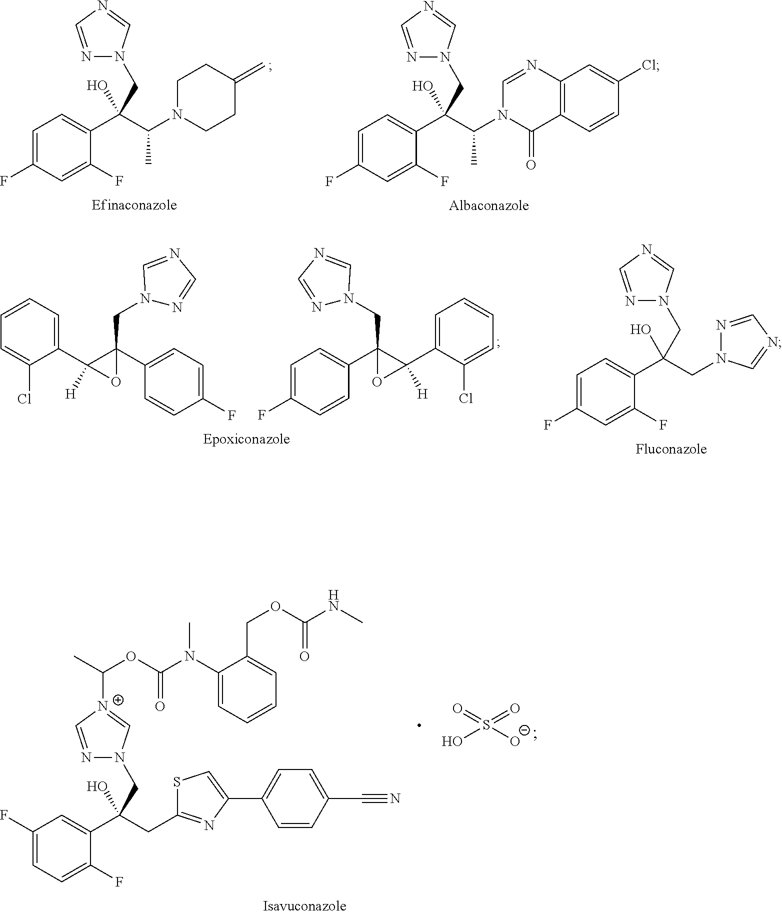

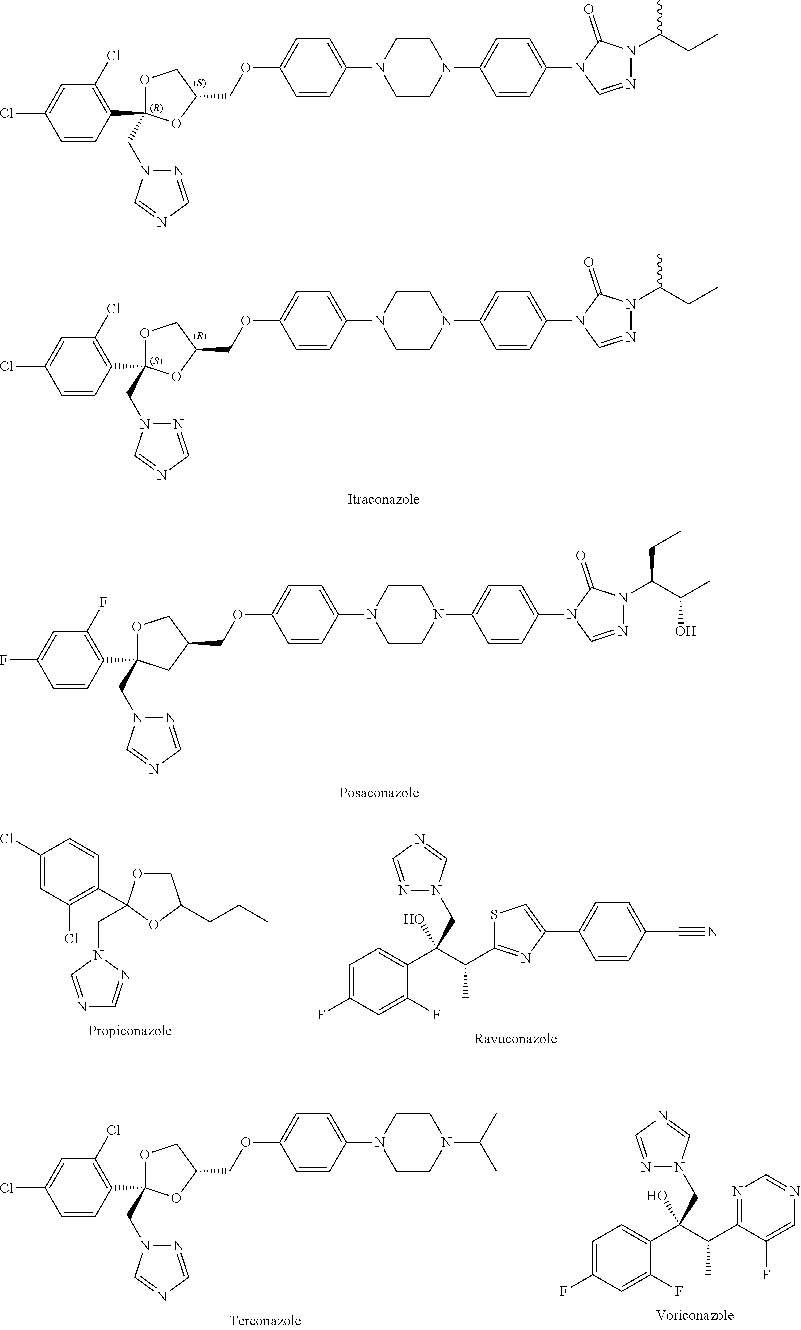

Accordingly, in a preferred embodiment, the azole antifungal agent is a triazole antifungal agent selected from albaconazole, efinaconazole, epoxiconazole, fluconazole, isavuconazole, itraconazole, posaconazole, propiconazole, ravuconazole, terconazole, voriconazole. In a preferred embodiment, the triazole antifungal agent is efinaconazole.

Efinaconazole compositions are known to the skilled person. In prior art compositions efinaconazole is formulated in an aqueous solution, comprising 10% w/v efinaconazole. A disadvantage of the compositions according to the prior art is that efinaconazole is prone to oxidation, specifically N-oxidation, and as such, the compositions of the prior art require an antioxidant, such as BHT.

The inventors surprisingly found that a composition comprising efinaconazole dissolved in a semifluorinated alkane shows improved stability against N-oxidation compared to an aqueous solution. Semifluorinated alkanes were known to improve the stability of compounds with more than one aliphatic double bonds, in which the aliphatic double bonds were prone to oxidation; see WO 2014/041071 A1. However, a stabilization of compounds prone to N-oxidation was unexpected.

It was further surprisingly found that a composition comprising efinaconazole dissolved in a semifluorinated alkane, shows effective penetration into nail material and a quick onset of action, namely showing quick inhibition of growth of a representative dermatophyte.

Semifluorinated alkanes are linear or branched alkanes some of whose hydrogen atoms have been replaced by fluorine. In a preferred embodiment, the semifluorinated alkanes (SFA's) used in the present invention are composed of at least one non-fluorinated hydrocarbon segment and at least one perfluorinated hydrocarbon segment. Particularly useful are SFA's which have one non-fluorinated hydrocarbon segment attached to one perfluorinated hydrocarbon segment, according to the general formula F(CF₂)ₙ(CH₂)ₘH, or two perfluorinated hydrocarbon segments separated by one non-fluorinated hydrocarbon segment, according to the general formula F(CF₂)ₙ(CH₂)ₘ(CF₂)ₒF.

Another nomenclature which is used herein refers to the above-mentioned SFA's having two or three segments as RFRH and RFRHRF, respectively, wherein R_{F} designates a perfluorated hydrocarbon segment, R_{H} designates a non-fluorinated segment. Alternatively, the compounds may be referred to as FₙHₘ and FₙHₘFₒ, respectively, wherein F means a perfluorated hydrocarbon segment, H means a non-fluorinated segment, and n, m and o is the number of carbon atoms of the respective segment. For example, F3H3 is used for perfluoropropylpropane. Moreover, this type of nomenclature is usually used for compounds having linear segments. Therefore, unless otherwise indicated, it should be assumed that F3H3 means 1-perfluoropropylpropane, rather than 2-perfluoropropylpropane, 1-perfluoroisopropylpropane or 2-perfluoroisopropylpropane.

Preferably, the semifluorinated alkanes according to the general formulas F(CF₂)ₙ(CH₂)ₘH and F(CF₂)ₙ(CH₂)ₘ (CF₂) OF have segment sizes ranging from n=2 to 20 carbon atoms, i.e. n, m and o are independently selected in the range from 2 to 20. SFAs which are useful in the context of the present invention are also described in EP-A 965 334, EP-A 965329 and EP-A 2110126, the disclosure of which documents is incorporated herein.

Accordingly, in one embodiment the invention relates to a composition as defined above, wherein the triazole antifungal is dissolved in the vehicle comprising a semifluorinated alkane. In some embodiments, the liquid vehicle consists of the semifluorinated alkane. In particular embodiments, the composition comprises a triazole antifungal dissolved in a semifluorinated alkane.

Any liquid semifluorinated is suitable for use in the liquid vehicle of the present invention. Preferred liquid semifluorinated alkanes are of the formula F(CF₂)ₙ(CH₂)ₘH, with n=4-8, m=4-10. Accordingly, in some embodiments of the invention, the semifluorinated alkane is of formula F(CF₂)ₙ(CH₂)ₘH, with n=4-8, m=4-10. In preferred embodiments, the semifluorinated alkane is selected from F4H5, F4H6, F4H8, F4H10, F6H6, F6H8, F6H10, preferably the semifluorinated alkane is selected from F4H8, F6H8, more preferably the semifluorinated alkane is F6H8. Liquid SFA's are chemically and physiologically inert, colourless and stable. Their typical densities range from 1.1 to 1.7 g/cm³, and their surface tension may be as low as 19 mN/m. SFA's of the RFRH type are insoluble in water but also somewhat amphiphilic, with increasing lipophilicity correlating with an increasing size of the non-fluorinated segment. Again, for practising the current invention, an SFA having a density of at least 1.2 g/cm³ should be selected.

The composition may comprise or consist of only the triazole antifungal and the liquid vehicle. The liquid vehicle may be comprised of the semifluorinated alkane and additional components. In some embodiments, the liquid vehicle comprises more than one semifluorinated alkane.

The semifluorinated alkane may be present in the composition at a concentration of:
i. up to 70, 75, 80, 85, 90, 91, 92, 93, 94, 95 or up to 97 vol %
ii. of between 60 to 90 vol %, 70 to 90 vol %, 80 to 90 vol %, 85 to 90 vol %, 65 to 95 vol %, 70 to 95 vol %, 80 to 95 vol % or between 85 to 95 vol %
iii. of about 65, 70, 75, 80, 85, 87, 89, 90, 91, 92, 93 or of about 95 vol %
iv. of at least 60, 70, 75, 80, 85, 87, 89, 90, 92, or at least 95 vol %

With respect to the present invention “vol %” or “% (v/v)” refers to the volume of the referenced compound compared to the total volume of the composition. 97 vol % of semifluorinated alkane would therefore refer to 97 ml of semifluorinated alkane per 100 ml of composition.

In some embodiments, the composition comprises further semifluorinated alkanes. If the composition comprises more than one semifluorinated alkane, it is preferred that one of the semifluorinated alkanes is a linear semifluorinated alkane and one semifluorinated alkane is a branched semifluorinated alkane.

In some embodiments, the composition additionally comprises 2-perfluorobutylpentan or 2-perfluorohexyloctane. In some embodiments of the invention, the composition as defined above comprises 2-perfluorohexyloctane. In a particular embodiment, the invention relates to a composition as defined above, wherein the composition comprises 2-perfluorohexyloctane, preferably at a concentration of
i. up to 0.25, 0.5, 0.75, 1.0, 1.5, 2.0 or up to 3 vol %
ii. up to 5, 4, 3, 2, 1 or up to 0.5 vol %,
iii. of between 0.25 to 5 vol %, 0.25 to 3 vol %, 0.25 to 2 vol %, 0.25 to 1.5 vol %, or between 0.25 to 1.0 vol %

The composition may comprise additional compounds. Preferably, the liquid vehicle comprises additional compounds. In some embodiments, the composition is free of additional pharmaceutically active compounds, in particular preferred embodiments the composition is free of additional antifungal agents, preferably the composition is free of a boron-containing antifungal agent, more preferably the composition is free of tavaborole. In some embodiments, the composition comprises additional pharmaceutically active compounds.

If the composition comprises additional compounds, it is preferred that the composition further comprises pharmaceutically acceptable excipients, such as co-solvents, oily materials and similar agents. In preferred embodiments of the invention, the liquid vehicle comprises a cosolvent and/or an oily material. A co-solvent should of course be selected in type, quality and amount such as to maintain the physiological tolerability of the formulation. Potentially suitable co-solvents include ethanol, acetone, ethyl acetate, isopropyl alcohol, DMF (N,N-dimethylformamide), DMSO (dimethyl sulfoxide), DMAC (N,N-dimethylacetamide), 1-Methyl-2-pyrrolidon (NMP), glycerol, propylene glycol, pentylene glycol, polyethylene glycol, liquid paraffin, triglyceride oils, silicon oils (i.e. linear and cyclic silicon oils), hydrofluorocarbons such as HFA 134a and/or HFA 227, and liquid mono- or diglycerides. Among these, ethanol, isopropyl alcohol, ethyl acetate, DMF, DMAC, DMSO and NMP are among the particularly preferred solvents.

In preferred embodiments the pharmaceutical composition comprises a cosolvent. In some embodiments, the cosolvent is selected from ethanol, 2-propanol, DMF (N,N-dimethylformamide), DMSO (dimethyl sulfoxide), DMAC (N,N-dimethylacetamide) and ethyl acetate, preferably the cosolvent is selected from 2-propanol, DMF, DMSO, DMAC, NMP and mixtures thereof; most preferably the cosolvent is 2-propanol.

Accordingly, in some embodiments, the invention relates to a composition as defined above, comprising a liquid vehicle comprising up to 25 vol % of a cosolvent selected from 2-propanol, DMF, DMSO, DMAC, NMP and mixtures thereof,

In some embodiments, the composition comprises an oily material. In some embodiments of the invention, the oily material is selected from squalane, MCT, silicon oils (i.e. linear or cyclic silicon oils),

An advantage of the presence of a cosolvent or an oily material is to increase the solubility of the triazole antifungals, or the possibility to adjust viscosity or other properties of the composition.

In a preferred embodiment the composition if free of further components, such as stabilisers, preservatives, surfactants, cosurfactants, colouring agents, thickeners (viscosity-increasing agents such as bentonite) fragrances and substances that may be irritant to the skin.

In a further preferred embodiment, the composition is free of one or more components selected from ethanol, 2-propanol, propylene glycol, ethyl acetate, amyl acetate and DMSO, more preferably the composition is free of one or more components selected from propylene glycol, ethyl acetate and amyl acetate.

In a preferred embodiment the composition if free of water.

A further advantage of the present invention is that, as noted above, that the composition as defined above is suitable to stabilize the triazole antifungal agent without the need for an antioxidant.

Accordingly, in some embodiments, the invention relates to a composition as defined above wherein the composition is free of antioxidants, preferably free of BHT, BHA, tocopherol.

The composition is further preferably free of ethanol. As such, the composition should be inflammable.

The composition according to the present invention can be a simpler composition than prior art compositions, reducing the number of ingredients. A reduced number of ingredients reduces the risk of adverse effects, such as skin irritation and potential allergic reactions.

The compositions according to the present invention require fewer compounds to achieve the effects compared to compositions of the prior art. As such, the invention relates in some preferred embodiments to a composition being free of one or more further components selected from
i. alcohol (preferably ethanol)
ii. penetration enhancer (preferably transcutol, isopropyl myristate)
iii. chelating agent (preferably EDTA, DTPA)
iv. wetting agent or surface tension lowering agent (preferably cyclomethicone)
v. pH adjusting agent (preferably citric acid)
vi. moisturizer (preferably C12-15alkyl lactate)
vii. emollient (preferably diisopropyl adipate)
viii. water
ix. preservative
x. surfactant
xi. ethanol, 2-propanol, propylene glycol, ethyl acetate, amyl acetate and DMSO, preferably propylene glycol, ethyl acetate, amyl acetate and DMSO (more preferably propylene glycol, ethyl acetate and amyl acetate)

Due to the fact that the composition is surprisingly suitable to stabilize efinaconazole, the composition of the invention is particularly suitable for efinaconazole. In preferred embodiments, the anti-fungal agent is efinaconazole and the composition comprises less than about 2, 1, 0.5, 0.4, 0.2, 0.1, 0.05 or less than 0.01% (w/v) of efinaconazole-N-oxid.

In a particular preferred embodiment, the invention relates to a composition, comprising or consisting of efinaconazole dissolved in a liquid vehicle comprising or consisting of 1-perfluorohexyloctane, and optionally a cosolvent selected from 2-propanol, DMF, DMSO, DMAC, and/or NMP.

In a particular preferred embodiment, the invention relates to a composition, comprising or consisting of efinaconazole dissolved in a liquid vehicle comprising or consisting of 1-perfluorohexyloctane, and optionally 2-propanol.

In a further embodiment, the invention relates to a composition, consisting of fluconazole dissolved in 1-perfluorohexyloctane, and optionally a cosolvent selected from 2-propanol, DMF, DMSO, DMAC, and/or NMP.

In a particular preferred embodiment, the invention relates to a composition, comprising or consisting of fluconazole dissolved in a liquid vehicle comprising or consisting of 1-perfluorohexyloctane, and optionally 2-propanol.

The inventors also found that a triazole antifungal provided in a composition according to the present invention can be used in quite substantial lower concentrations compared to prior art aqueous compositions to achieve an equivalent or better reduction or kill of fungal manifestations.

Accordingly, in some embodiments the invention relates to a composition as defined above, wherein the antifungal agent is present at a concentration of:
i. up to 20, 10, 5, 2, 1.5, 1, 0.7, 0.6, 0.5, 0.4 or 0.3% (w/v)
ii. of between 0.2 to 10% (w/v), 0.2 to 5% (w/v), 0.2 to 3% (w/v), 0.3 to 1.5% (w/v), 0.3 to 1% (w/v), 0.4 to 1.5% (w/v), 0.4 to 1% (w/v), 0.5 to 1.5% (w/v), 0.5 to 1.0% (w/v), 0.7 to 1.5% (w/v), 0.7 to 1.0% (w/v), or 0.1 to 2% (w/v), or 0.1 to 1% (w/v)
iii. of about 10, 5, 3, 1.5, 1, 0.9, 0.8, 0.7, 0.6, 0.5, 0.4, 0.3 or of about 0.2% (w/v)
iv. of at least 0.2, 0.3, 0.4, 0.5, 0.6, 0.7, 0.8, 0.9, 1.0 or of at least 1.5% (w/v)
v. of between 0.5 to 2.5% (w/v), 0.75 to 2.25% (w/v), 1.0 to 2% (w/v), 1.2 to 1.8% (w/v) or 1.4 to 1.6% (w/v)

In a particular embodiment, the invention relates to a composition as defined above, comprising or consisting of about 0.5 to 2.5% (w/v) (or 0.5 to 1.0% (w/v), or 0.1 to 2% (w/v) or 0.1 to 1% (w/v), or 0.5 to 2.5% (w/v), or 0.75 to 2.25% (w/v), or 1.0 to 2% (w/v), or 1.2 to 1.8% (w/v) or 1.4 to 1.6% (w/v)) efinaconazole dissolved in 98 to 99.5% (w/v) (or 99 to 99.5% (w/v)) of a vehicle comprising or consisting of 1-perfluroohexyloctane and 2-propanol. In a further embodiment, the invention relates to a composition as defined above, comprising or consisting of up to about 0.5 to 2.5% (w/v) (or 0.5 to 1.0% (w/v), or 0.1 to 2% (w/v) or 0.1 to 1% (w/v), or 0.5 to 2.5% (w/v), or 0.75 to 2.25% (w/v), or 1.0 to 2% (w/v), or 1.2 to 1.8% (w/v) or 1.4 to 1.6% (w/v)) efinaconazole dissolved in 97.5 to 99.5% (w/v) (or 99 to 99.5% (w/v)) of a vehicle comprising or consisting of 1-perfluorohexyloctane and a cosolvent selected from 2-propanol, DMF, DMSO, DMAC, and/or NMP.

In a particular embodiment, the invention relates to a composition as defined above, comprising or consisting of about 0.5 to 2.5% (w/v) (or 0.5 to 1.0% (w/v), or 0.1 to 2% (w/v) or 0.1 to 1% (w/v), or 0.5 to 2.5% (w/v), or 0.75 to 2.25% (w/v), or 1.0 to 2% (w/v), or 1.2 to 1.8% (w/v) or 1.4 to 1.6% (w/v)) efinaconazole dissolved in 98 to 99.5% (v/v) (or 99 to 99.5% (v/v)) of a vehicle comprising or consisting of 1-perfluroohexyloctane and 2-propanol. In a further embodiment, the invention relates to a composition as defined above, comprising or consisting of up to about 0.5 to 2.5% (w/v) (or 0.5 to 1.0% (w/v), or 0.1 to 2% (w/v) or 0.1 to 1% (w/v), or 0.5 to 2.5% (w/v), or 0.75 to 2.25% (w/v), or 1.0 to 2% (w/v), or 1.2 to 1.8% (w/v) or 1.4 to 1.6% (w/v)) efinaconazole dissolved in 97.5 to 99.5% (v/v) (or 99 to 99.5% (v/v)) of a vehicle comprising or consisting of 1-perfluorohexyloctane and a cosolvent selected from 2-propanol, DMF, DMSO, DMAC, and/or NMP.

In a further embodiment, the composition comprises of consists of 0.1% (w/v) efinaconazole (or 0.2% (w/v), or 0.5% (w/v), or 1.0% (w/v), or 1.25% (w/v), or 1.5% (w/v), or 1.75% (w/v) or 2% (w/v) or 2.25% (w/v), or 2.5% (w/v) efinaconazole), about 90% (v/v) F6H8 and up to or equal to 10% (v/v) 2-propanol. In a particular embodiment the invention relates to a composition, consisting of up to 0.7% (w/v) efinaconazole and 1-perfluorohexyloctane, or a composition, consisting of 0.7% (w/v) efinaconazole and 1-perfluorohexyloctane.

In one embodiment, the composition comprises or consist of 1-2% (w/v) efinaconazole dissolved in a vehicle comprising or consisting of 1-perfluorhexyloctane and 2-propanol, preferably the composition comprises or consist of 1-2% (w/v) efinaconazole dissolved in a vehicle comprising or consisting of 90% (v/v) 1-perfluorhexyloctane and 10% (v/v) 2-propanol.

In a particular embodiment, the composition comprises or consist of 1% (w/v) efinaconazole dissolved in a vehicle comprising or consisting of 1-perfluorhexyloctane and 2-propanol, preferably the composition comprises or consist of 1% (w/v) efinaconazole dissolved in a vehicle comprising or consisting of 90% (v/v) 1-perfluorhexyloctane and 10% (v/v) 2-propanol.

In a further embodiment, the composition comprises or consist of 2% (w/v) efinaconazole dissolved in a vehicle comprising or consisting of 1-perfluorhexyloctane and 2-propanol, preferably the composition comprises or consist of 2% (w/v) efinaconazole dissolved in a vehicle comprising or consisting of 90% (v/v) 1-perfluorhexyloctane and 10% (v/v) 2-propanol.

In a further aspect, the invention relates to the uses of the composition, in particular pharmaceutical uses and applications of the composition as defined above. The compositions are particularly useful in the treatment of fungal infections of the skin (mycosis) and of the nail (onychomycosis), in particular the compositions of the inventions are effective in the treatment of onychomycosis of the nails, specifically fingernails and/or toenails. Further, the compositions are useful in the treatment of fungal infections of the eye, in particular the compositions of the inventions are effective in the treatment of fungal infections of the cornea (keratitis), or any other fungal infection of the eye caused by Fusarium and Aspergillus species.

In a further aspect, the compositions of the inventions are effective in the treatment of vaginal fungal infections, in particular composition of the invention comprising the fluconazole are effective in the treatment of vaginal yeast infection, also known as candidal vulvovaginitis and vaginal thrush.

Accordingly, in one embodiment, the invention relates to a composition as defined above for use as medicine. In a particular embodiment, the invention relates to the use of a composition as defined above in the manufacture of a medicine.

Said medicine is preferably a medicine for the treatment of a fungal infection of the skin, in particular a fungal infection of finger or toenails. Preferably, said medicine is a medicine for treatment of onychomycosis of finger or toenails.

Said medicine is also preferably a medicine for the treatment of a fungal infection of the eye, in particular a fungal infection of the cornea. Preferably, said medicine is a medicine for treatment of fungal infection of the eye caused by Fusarium and Aspergillus species.

Accordingly, in one embodiment, said medicine is for treating, preventing of reducing growth of a fungus affecting the skin or the ye, preferably a fungus affecting the nails, such as the finger and/or toenails of a subject or a fungus affecting the cornea.

A major advantage of compositions according to the invention is that the composition, once applied to the affected nails, will dry in significantly shorter time than other compositions, thus improving patient compliance.

Further, the compositions according to the invention comprising efinaconazole present as odor-less liquids, while the commercial efinaconazole formulation exhibit a highly unpleasant, acrid smell, that also limits patient compliance.

In another aspect, the invention relates to a composition as defined above for use in treating, preventing or reducing growth of a fungus selected from yeasts (i.e. Candida albicans) or moulds. Accordingly, in some embodiments, the invention relates to a composition as defined above, for use in the treatment of a fungal infection of the skin, preferably for use in the treatment of a fungal infection of the nails (onychomycosis), preferably in the treatment of onychomycosis of the fingernails and/or the toenails.

In another aspect, the invention relates to a composition as defined above for use in treating, preventing or reducing growth of a fungus selected from Fusarium and Aspergillus species. Accordingly, in some embodiments, the invention relates to a composition as defined above, for use in the treatment of a fungal infection of the eye, preferably for use in the treatment of a fungal infection of the cornea (keratitis), more preferably in the treatment of a fungal infection of the eye caused by Fusarium and Aspergillus species.

In a further aspect, the invention relates to a method for the treatment of a fungal infection of the skin, preferably a method for treatment of the nail, more preferably a method for the treatment of onychomycosis, particularly for the treatment of onychomycosis of the fingernails and/or the toenails, the method comprising administering a composition as defined above to the nail.

The composition is preferably administered directly to the affected nails. The composition may be applied by any suitable way. Accordingly, in one embodiment, the invention relates to a composition as defined above, wherein the composition is topically administered to a nail (fingernail or toenail) of the subject.

In a preferred embodiment the composition is administered by use of a sponge applicator, dropper, brush applicator, pen or a pipette to the fingernail and/or the toenail.

However, in one embodiment the composition is not applied with a brush or brush applicator.

The composition is preferably topically administered to cover the nail, the folds, the bed, the hyponychium and/or the undersurface of the plate of a toenail and/or a fingernail.

In some embodiments of the invention the composition is topically administered by non-contact application (without touching) to a nail (fingernail or toenail) of the subject, preferably by dropping one or more single drops of the liquid composition to the surface of the nail (fingernail or toenail) of the subject. In a particular embodiment, the composition is topically administered to cover the nail, the folds, the bed, the hyponychium and/or the undersurface of the plate of a toenail and/or a fingernail by dropping one or more single drops of the liquid composition to the (central) surface of the nail. It was surprisingly found that the compositions described above are characterised by superior spreading behaviour, which effectuates that single drops of the composition, when administered topically to the surface of a nail, such as a finger nail or toe nail, readily spread-without mechanical distribution-even to remote regions of the nail, such as the folds, the bed, the hyponychium and/or the undersurface of the plate of a toenail and/or a fingernail.

In some embodiments, the composition is topically administered by dropping one single drop to the surface of the nail (fingernail or toenail) of a subject,
i. without contacting (or touching) the nail, the folds, the bed, the hyponychium and/or the undersurface of the plate of a toenail and/or a fingernail and/or
ii. without manually distributing the composition to one or more remote regions of the toenail and/or a fingernail selected from the folds, the bed, the hyponychium and/or the undersurface of the plate.

In a further aspect, the invention relates to a method for the treatment of a fungal infection of the eye, preferably a method for treatment of the eye, more preferably a method for the treatment of a fungal infection of the cornea, particularly for the treatment of keratitis caused by fungal infection, in particular caused by Fusarium or Aspergillus species, the method comprising administering a composition as defined above to the eye.

The composition is preferably administered directly to the affected eye tissue, preferably to the affected cornea. The composition may be applied topically to the affected eye tissue, such as the cornea, preferably by use of an eyedrop. Accordingly, in one embodiment, the invention relates to a composition as defined above, wherein the composition is topically administered to the eye (i.e. eye surface, such as the cornea and/or the conjunctiva) of the subject, preferably by dropping one or more single drops of the liquid composition to the (central) surface of the affected eye, i.e. by dropping one or more single drops of the liquid composition to the surface of the cornea and/or conjunctiva.

In a preferred embodiment the composition is administered by use of an eyedropper, to the eye or the eye surface, in particular the composition is administered by one or more single drops of the liquid composition to the cornea and/or the conjunctiva.

In a further embodiment, the invention relates to a kit comprising a composition as defined above, and a container for holding the composition, wherein the container is adapted for topical administration of the composition to the skin, preferably to the nail, the folds, the bed, the hyponychium and/or the undersurface of the plate of a toenail and/or a fingernail of a subject.

In some embodiments, the invention relates to a kit comprising a composition as defined above, and instructions for use in the treatment of fungal infections of the skin and/or the nail.

In a further embodiment, the invention relates to a kit comprising a composition as defined above, and a container for holding the composition, wherein the container is adapted for topical administration of the composition to the eye, preferably the container is adapted for topical administration of the composition by one or more single drops of a the liquid composition to the eye (or to an eye surface, such as the cornea and/or conjunctiva) of a subject. Preferably, the container is an eyedropper that is adapted to dropping single drops of the liquid composition to the eye, preferably the eyedropper that is adapted to dropping single drops of 9-13 μl, 10-12 μl or 11 μl volume of the liquid composition to the eye.

In some embodiments, the invention relates to a kit comprising a composition as defined above, and instructions for use in the treatment of fungal infections of the eye an ophthalmic tissue and/or the cornea.

In an additional aspect, the invention relates to a composition comprising terbinafine (free base; CAS No. 91161-71-6) or a pharmaceutically acceptable salt thereof (i.e. hydrochloride salt CAS No. 78628-80-5) and a semifluorinated alkane.

In a preferred embodiment the composition comprises terbinafine and a semifluorinated alkane selected from the group consisting of F4H4, F4H5, F4H6, F4H8, F6H2, F6H4, F6H6, F6H8, F6H10. In an even more preferred embodiment, the semifluorinated alkane is F6H8.

The composition may optionally comprise one or more pharmaceutically acceptable cosolvent, preferably selected from ethanol, 2-propanol, isopropyl myristate, DMSO, mineral oil and squalane, more preferably the cosolvent is selected from ethanol and 2-propanol.

The composition comprises terbinafine at a concentration of 1-20 mg/ml, 1-15 mg/ml, 1-10 mg/ml, 5-15 mg/ml, 5-10 mg/ml or of 10 mg/ml.

In a preferred embodiment, the pharmaceutical composition is a composition of low complexity, a composition consisting of only 2 components, namely terbinafine dissolved in a semifluorinated alkane selected from F6H8 and F4H5. More preferably, the pharmaceutical composition is a liquid solution consisting of terbinafine and F6H8. Preferred is a pharmaceutical composition consisting of 1-15 mg/ml, 1-10 mg/ml, 5-15 mg/ml, 5-10 mg/ml or of 10 mg/ml terbinafine dissolved in F6H8.

In a further preferred embodiment, the liquid pharmaceutical composition consists of terbinafine dissolved in F6H8 and up to 10 wt % of a cosolvent selected from ethanol or 2-propanol. Herein, the pharmaceutical composition consists of 1-15 mg/ml, 1-10 mg/ml, 5-15 mg/ml, 5-10 mg/ml or of 10 mg/ml terbinafine dissolved in F6H8 and up to 10 wt % of a cosolvent selected from ethanol or 2-propanol.

The compositions comprising terbinafine are particularly useful in the topical treatment of fungal infections of the skin (mycosis), of the nail (onychomycosis) or the eye (i.e. fungal infections of the cornea, such as keratitis), in particular the compositions of the inventions are effective in the topical treatment of onychomycosis of the nails, specifically finger nails and/or toe nails or are effective in the topical treatment of fungal infections of the cornea, specifically caused by Fusarium or Aspergillus species.

The application further relates to the following numbered items:
1. A liquid composition comprising an azole antifungal agent and a vehicle comprising a semifluorinated alkane.
2. The composition according to item 1, wherein the azole antifungal is a triazole antifungal agent selected from albaconazole, efinaconazole, epoxiconazole, fluconazole, isavuconazole, itraconazole, posaconazole, propiconazole, ravuconazole, terconazole, voriconazole.
3. The composition according to any of the preceding items, wherein the antifungal agent is selected from efinaconazole or fluconazole, preferably the antifungal agent is efinaconazole, more preferably the antifungal agent is efinaconazole and the composition does not comprise a further active ingredient, most preferably the antifungal agent is efinaconazole and the composition does not comprise tavaborole.
4. The composition according to any of the preceding items, wherein the triazole antifungal is dissolved in the vehicle comprising a semifluorinated alkane.
5. The composition according to any of the preceding items, wherein the semifluorinated alkane is of formula F(CF₂)ₙ(CH₂)ₘH, with n=2-8, m=2-10, preferably the semifluorinated alkane is of formula F(CF₂)ₙ(CH₂)ₘH, with n=4-8, m=4-10.
6. The composition according to any of the preceding items, wherein the semifluorinated alkane is selected from F4H5, F4H6, F4H8, F4H10, F6H6, F6H8, F6H10, preferably the semifluorinated alkane is selected from F4H8, F6H8, more preferably the semifluorinated alkane is F6H8.
7. The composition according to any preceding item, wherein the semifluorinated alkane is present at a concentration of:
   i. up to 70, 75, 80, 85, 90, 91, 92, 93, 94, 95 or up to 97% (v/v)
   ii. of between 60 to 90% (v/v), 70 to 90% (v/v), 80 to 90% (v/v), 85 to 90% (v/v), 65 to 95% (v/v), 70 to 95% (v/v), 80 to 95% (v/v) or between 85 to 95% (v/v)
   iii. of about 65, 70, 75, 80, 85, 87, 89, 90, 91, 92, 93 or of about 95% (v/v)
   iv. of at least 60, 70, 75, 80, 85, 87, 89, 90, 92, or at least 95% (v/v)
8. The composition according to any preceding item, wherein the vehicle comprises a cosolvent or an oily material.
9. The composition according to item 8, wherein the cosolvent is selected from ethanol, 2-propanol, DMF (N,N-dimethylformamide), DMSO (dimethyl sulfoxide), DMAC (N,N-dimethylacetamide), NMP (1-Methyl-2-pyrrolidon)
10. The composition according to item 8, wherein the oily material is selected from squalane, MCT, or silicon oils
11. The composition according to any preceding item, wherein the antifungal agent is present at a concentration of:
   i. up to 20, 10, 5, 2, 1, 0.7, 0.6, 0.5, 0.4 or 0.3% (w/v)
   ii. of between 0.2 to 10% (w/v), 0.2 to 5% (w/v), 0.2 to 3% (w/v), 0.3 to 1% (w/v), 0.4 to 1% (w/v), 0.5 to 1.0% (w/v), 0.7 to 1.0% (w/v)
   iii. of about 10, 5, 3, 1, 0.9, 0.8, 0.7, 0.6, 0.5, 0.4, 0.3 or of about 0.2% (w/v)
   iv. of at least 0.2, 0.3, 0.4, 0.5, 0.6, 0.7, 0.8, 0.9, or of at least 1.0% (w/v)
   v. of between 0.5 to 2.5% (w/v), 0.75 to 2.25% (w/v), 1.0 to 2% (w/v), 1.2 to 1.8% (w/v) or 1.4 to 1.6% (w/v)
12. The composition according to any preceding item, being not inflammable.
13. The composition according to any preceding item, being free of antioxidants, preferably selected from BHT, BHA, tocopherol
14. The composition according to any preceding item, wherein the composition comprises efinaconazole and less than about 2, 1, 0.5, 0.4, 0.2 or less than 0.1% (w/v) of efinaconazole-N-oxid
15. The composition according to any preceding item, being free of one or more further components selected from
   i. alcohol (preferably ethanol)
   ii. penetration enhancer (preferably transcutol, isopropyl myristate)
   iii. chelating agent (preferably EDTA, DTPA)
   iv. wetting agent or surface tension lowering agent (preferably cyclomethicone)
   v. pH adjusting agent (preferably citric acid)
   vi. moisturizer (preferably C12-15alkyl lactate)
   vii. emollient (preferably diisopropyl adipate)
   viii. water
   ix. preservative
   x. surfactant
   xi. ethanol, 2-propanol, propylene glycol, ethyl acetate, amyl acetate and DMSO (preferably propylene glycol, ethyl acetate and amyl acetate)
16. The composition according to any preceding item, being free of one or more further components selected from ethanol, anhydrous citric acid, butylated hydroxytoluene, C12-15alkyl lactate, cyclomethicone, diisopropyl adipate, disodium edetate and water.
17. The composition according to any preceding item, wherein the composition comprises 2-perfluorohexyloctane, preferably at a concentration of
   i. up to 0.25, 0.5, 0.75, 1.0, 1.5, 2.0 or up to 3% (v/v)
   ii. up to 5, 4, 3, 2, 1 or up to 0.5% (v/v),
   iii. of between 0.25 to 5% (v/v), 0.25 to 3% (v/v), 0.25 to 2% (v/v), 0.25 to 1.5% (v/v), or between 0.25 to 1.0% (v/v)
18. The composition according to any preceding item, comprising or consisting of efinaconazole dissolved in vehicle comprising or consisting of 1-perfluorohexyloctane, and optionally 2-propanol (or ethanol).
19. The composition according to any preceding item, comprising or consisting of about 0.1 to 2% (w/v), or 0.5 to 2% (w/v) or 1.0 to 2% (w/v), or 1.5 to 2% (w/v), or 1.75 to 2.0% (w/v), or 1.2 to 1.8% (w/v) or 1.4 to 1.6% (w/v) (or 0.5 to 1.0% (w/v)) efinaconazole dissolved in 98 to 99.5% (v/v) (or 99 to 99.5% (v/v)) of a vehicle comprising or consisting of 1-perfluroohexyloctane and 2-propanol, preferably comprising or consisting of about 0.1 to 2% (w/v), or 0.5 to 2% (w/v) or 1.0 to 2% (w/v), or 1.5 to 2% (w/v), or 1.75 to 2.0% (w/v), or 1.2 to 1.8% (w/v) or 1.4 to 1.6% (w/v) (or 0.5 to 1.0% (w/v)) efinaconazole dissolved a vehicle comprising or consisting of at least 90% (v/v) 1-perfluroohexyloctane and up to 10% (v/v) 2-propanol.
20. The composition according to any preceding item, comprising or consisting of:
   i.) 1% (w/v) efinaconazole dissolved in a vehicle comprising or consisting of at least 90% (v/v) 1-perfluroohexyloctane and up to 10% (v/v) 2-propanol
   ii.) 2% (w/v) efinaconazole dissolved in a vehicle comprising or consisting of at least 90% (v/v) 1-perfluroohexyloctane and up to 10% (v/v) 2-propanol
   iii.) 1 to 2% (w/v) efinaconazole dissolved in a vehicle comprising or consisting of at least 90% (v/v) 1-perfluroohexyloctane and up to 10% (v/v) 2-propanol.
21. The composition according to any preceding item, comprising or consisting of about 0.1 to 2% (w/v) or 0.1 to 1% (w/v) or 1.0 to 2% (w/v), or 1.5 to 2% (w/v), or 1.75 to 2.0% (w/v), or 1.2 to 1.8% (w/v) or 1.4 to 1.6% (w/v) (or 0.5 to 2.0% (w/v)) efinaconazole dissolved in at least 98 to 99.5% (v/v) (or at least 99 to 99.5% (v/v)) of a vehicle comprising or consisting of 1-perfluroohexyloctane and ethanol, preferably comprising or consisting of about 0.1 to 2% (w/v), or 0.5 to 2% (w/v) or 1.0 to 2% (w/v), or 1.5 to 2% (w/v), or 1.75 to 2.0% (w/v), or 1.2 to 1.8% (w/v) or 1.4 to 1.6% (w/v) (or 0.5 to 1.0% (w/v)) efinaconazole dissolved a vehicle comprising or consisting of at least 98.5% (v/v) 1-perfluroohexyloctane and up to 1.5% (v/v) ethanol.
22. The composition according to any preceding item, comprising or consisting of up to about 0.5 to 2.0% (w/v) or 1.0 to 2% (w/v), or 1.5 to 2% (w/v), or 1.75 to 2.0% (w/v), or 1.2 to 1.8% (w/v) or 1.4 to 1.6% (w/v) (or 0.5 to 1.0% (w/v)) efinaconazole dissolved in 98 to 99.5% (w/v) (or 99 to 99.5% (w/v)) of a vehicle comprising or consisting of 1-perfluorohexyloctane, 2-propanol and optionally one or more of DMF (N,N-dimethylformamide), DMSO (dimethyl sulfoxide), DMAC (N,N-dimethylacetamide) and NMP (1-Methyl-2-pyrrolidon.
23. The composition according to any preceding item, comprising or consisting of up to about 0.5 to 2.0% (w/v) or 1.0 to 2% (w/v), or 1.5 to 2% (w/v), or 1.75 to 2.0% (w/v), or 1.2 to 1.8% (w/v) or 1.4 to 1.6% (w/v) (or 0.5 to 1.0% (w/v)) efinaconazole dissolved in 98 to 99.5% (v/v) (or 99 to 99.5% (v/v)) of a vehicle comprising or consisting of 1-perfluorohexyloctane, 2-propanol and optionally one or more of DMF (N,N-dimethylformamide), DMSO (dimethyl sulfoxide), DMAC (N,N-dimethylacetamide) and NMP (1-Methyl-2-pyrrolidon.
24. The composition according to any preceding item, comprising a vehicle with up to 10% (v/v) of a cosolvent selected from ethanol, 2-propanol, MCT, DMF (N,N-dimethylformamide), DMSO (dimethyl sulfoxide), DMAC (N,N-dimethylacetamide) and NMP (1-Methyl-2-pyrrolidon.
25. The composition according to any preceding item, consisting of up 1.5% (w/v) efinaconazole, up to 85% (w/v) of 1-perfluorohexyloctane and less than or equal to 15% (w/v) of one or more cosolvents selected from 2-propanol, DMF (N,N-dimethylformamide), DMSO (dimethyl sulfoxide), DMAC (N,N-dimethylacetamide) and NMP (1-Methyl-2-pyrrolidon, preferably consisting of up to 1.5% (w/v) efinaconazole, up to 90% (w/v) of 1-perfluorohexyloctane and less than or equal to 10% (w/v) of one or more cosolvents selected from 2-propanol, DMF (N,N-dimethylformamide), DMSO (dimethyl sulfoxide), DMAC (N,N-dimethylacetamide) and NMP (1-Methyl-2-pyrrolidon.
26. The composition according to any preceding item, consisting of up 1.5% (w/v) efinaconazole, up to 85% (w/v) of 1-perfluorohexyloctane and less than or equal to 15% (v/v) of one or more cosolvents selected from 2-propanol, DMF (N,N-dimethylformamide), DMSO (dimethyl sulfoxide), DMAC (N,N-dimethylacetamide) and NMP (1-Methyl-2-pyrrolidon, preferably consisting of up to 1.5% (v/v) efinaconazole, up to 90% (v/v) of 1-perfluorohexyloctane and less than or equal to 10% (v/v) of one or more cosolvents selected from 2-propanol, DMF (N,N-dimethylformamide), DMSO (dimethyl sulfoxide), DMAC (N,N-dimethylacetamide) and NMP (1-Methyl-2-pyrrolidon.
27. The composition according to any preceding item, for use as medicine.
28. The composition for use according to item 27, for use in the treatment of a fungal infection of the skin, preferably for treatment of a fungal infection of the nail (onychomycosis), more preferably in the treatment of onychomycosis of the fingernails and/or the toenails.
29. The composition for use according to item 27 to 28, wherein the composition is topically administered to a nail (fingernail or toenail) of the subject.
30. The composition for use according to item 27 to 29, wherein the composition is administered by use of a sponge applicator, dropper, pen or a pipette to the fingernail and/or the toenail.
31. The composition for use according to item 27 to 30, wherein the composition is not administered by a brush applicator.
32. The composition for use according to item 27 to 31, wherein the composition is topically administered to cover the nail, the folds, the bed, the hyponychium and/or the undersurface of the plate of a toenail and/or a fingernail.
33. The composition for use according to items 27 to 32, wherein the composition is topically administered by non-contact application to a nail (fingernail or toenail) of the subject, preferably by dropping one or more single drops of the liquid composition to the surface of the nail (fingernail or toenail) of the subject.
34. The composition for use according to items 27 to 33, wherein the composition is topically administered to cover the nail, the folds, the bed, the hyponychium and/or the undersurface of the plate of a toenail and/or a fingernail by dropping one or more single drops of the liquid composition to the (central) surface of the nail.
35. The composition for use according to items 27 to 33, wherein the composition is topically administered by dropping one single drop to the surface of the nail (fingernail or toenail) of a subject,
   i. without contacting the nail, the folds, the bed, the hyponychium and/or the undersurface of the plate of a toenail and/or a fingernail and/or
   ii. without manually distributing the composition to one or more remote regions of the toenail and/or a fingernail selected from the folds, the bed, the hyponychium and/or the undersurface of the plate, and/or
   iii. resulting in quick drying, preferably within less than 20, 10 min or less than 5 minutes at room temperature
36. The composition for use according to item 27, for use in the treatment of a fungal infection of the eye, preferably for treatment of a fungal infection of the cornea (keratitis), more preferably in the treatment of a fungal infection of the cornea (keratitis) caused by Fusarium or Aspergillus species.
37. The composition for use according to item 36, wherein the composition is topically administered to the eye (i.e. eye surface, such as the cornea and/or the conjunctiva) of the subject, preferably by dropping one or more single drops of the liquid composition to the (central) surface of the affected eye, more preferably by dropping one or more single drops of the liquid composition to the surface of the cornea and/or conjunctiva.
38. A kit comprising the composition as defined in any of the items 1 to 24, and a container for holding the composition, wherein the contained is adapted for topical administration of the composition to the nail, the folds, the bed, the hyponychium and/or the undersurface of the plate of a toenail and/or a fingernail of a subject.
39. A kit comprising the composition as defined in any of the items 1 to 26, and a container for holding the composition, wherein the contained is adapted for topical administration of the composition to the eye or an ophthalmic tissue of a subject.
40. A liquid composition comprising terbinafine or a pharmaceutically acceptable salt thereof, a semifluorinated alkane, and optionally a pharmaceutically acceptable cosolvent.
41. The composition according to item 40, wherein the semifluorinated alkane is selected from the group consisting of F4H4, F4H5, F4H6, F4H8, F6H2, F6H4, F6H6, F6H8, F6H10, preferably the semifluorinated alkane is selected from F4H5 and F6H8, more preferably the semifluorinated alkane is F6H8.
42. The composition according to any one of items 40 or 41, wherein the terbinafine or a pharmaceutically acceptable salt thereof is present at a concentration of 1-20 mg/ml, preferably at a concentration of 1-10 mg/ml, more preferably terbinafine is present at 10 mg/ml.
43. The composition according to any one of items 40 to 42, wherein the composition is in form of a solution.
44. The composition according to any one of items 40 to 43, wherein the composition comprises an excipient selected from the group consisting of ethanol, 2-propanol, isopropyl myristate, DMSO, mineral oil and squalane, more preferably the cosolvent is selected from ethanol and 2-propanol.
45. The composition according to any one of items 40 to 44, wherein the composition consists of terbinafine and semifluorinated alkane selected from F4H5 and F6H8, preferably the semifluorinated alkane is F6H8.
46. The composition according to any one of items 40 to 45, wherein the composition consists of 1-15 mg/ml, 1-10 mg/ml, 5-15 mg/ml, 5-10 mg/ml or of 10 mg/ml terbinafine dissolved in F6H8.
47. The composition according to any one of items 40 to 46, for use in medicine.
48. The composition according to item 47, for use in the topical treatment of a fungal infection, preferably for use in the treatment of a fungal infection of the skin or a nail (or the eye), more preferably for use in the treatment of onychomycosis (or a fungal infection of the cornea).
49. A method for treatment of fungal infection comprising topically administering the composition according to items 40-46 to a subject in need thereof, preferably wherein the composition is topically administered to the skin or a nail (or an ophthalmic tissue) of the subject.
50. The method of treatment according to item 49, wherein the subject suffers from onychomycosis or a fungal infection of the cornea.
51. A kit comprising the composition as defined in any of the items 40 to 46, and a container for holding the composition, wherein the contained is adapted for topical administration to the nail, the folds, the bed, the hyponychium and/or the undersurface of the plate of a toenail and/or a fingernail of a subject or wherein the contained is adapted for topical administration of the composition to the eye or an ophthalmic tissue of a subject.

The following examples serve to illustrate the invention, however should not be understood as restricting the scope of the invention.

### EXAMPLES

### Example 1

### Preparation of a Solution of Efinaconazole in F6H8

14 mg efinaconazole (purity >99%) is weighed in a 5-ml glass vial; 2 ml F6H8 (Novaliq, purity >99%) is added into the vial by using a pipette, closed with aluminium crimp cap and shaken for 4 hours at 280 mpm, at RT. The resulting 7 mg/ml composition of efinaconazole in F6H8 presents as clear solution.

### Example 2

20 mg efinaconazole (purity >99%) is weighed in a 5-ml glass vial; the needed volume of the cosolvent is added into the vial by using a pipette; the vial is shaken by hand gentle; the needed volume of F6H8 (Novaliq, purity >99%) is added by using a pipette, closed with aluminium crimp cap and shaken for 4 hours at 280 mpm, at RT. The resulting composition of efinaconazole in F6H8/cosolvent presents as clear solution.

Following the procedure described in the Examples 1 and 2, the following compositions were prepared (Table 1):

TABLE 1
Efinaconazole compositions

| Efinaconazole | semifluorinated | | |
|---|---|---|---|
| concentration | alkane | Cosolvent(s) | Appearance |
| 7 mg/mL | F6H8 | | clear solution |
| 10 mg/mL | F6H8 (90 vol %) | 2-propanol (10 vol %) | clear solution |
| 25 mg/mL | F6H8 (75 vol %) | 2-propanol (25 vol %) | clear solution |
| 15 mg/mL | F6H8 (85 vol %) | 2-propanol (10 vol %) | clear solution |
| | | 1-Methyl-2-pyrrolidon (5 vol %) | |
| 15 mg/mL | F6H8 (85 vol %) | 2-propanol (10 vol %) | clear solution |
| | | N,N-Dimethyl formamide (5 vol %) | |
| 15 mg/mL | F6H8 (85 vol %) | 2-propanol (10 vol %) | clear solution |
| | | N,N-Dimethyl acetamide (5 vol %) | |
| 15 mg/mL | F6H8 (85 vol %) | 2-propanol (10 vol %) | clear solution |
| | | Ethyl acetate (5 vol %) | |
| 15 mg/mL | F4H5 (90 vol %) | 2-propanol (10 vol %) | clear solution |
| 15 mg/mL | F4H10 (90 vol %) | 2-propanol (10 vol %) | clear solution |

### Example 3: In Vitro Nail Penetration/Inhibition Model

Three test products containing 0.1% to 10% efinaconazole were tested in vitro for their capabilities to inhibit growth of a representative dermatophyte (T. interdigitale) that is known to cause onychomycosis and tinea pedis in humans:
(a) 0.1% efinaconazole, dissolved in a mixture of F6H8 and 2-propanol (10:1 v/v %), non-aqueous (8-9),
(b) 1.0% efinaconazole, dissolved in a mixture of F6H8 and 2-propanol (9:1 v/v %), non-aqueous (4-7),
(c) 10% efinaconazole, aqueous commercial product (“Jublia”; Bausch Health, Bridgewater, USA) (10-11).

As reference products, DMSO was utilized as negative control (1), terbinafine (100 μg/ml in DMSO) as positive control (2) and F6H8 as vehicle control (3). The non-aqueous efinaconazole formulations (0.1% and 1%) (4-9) did not contain any antioxidant, presented as colourless solutions, and did not show any discoloration during performance of the testing.

Bovine Hoof Sheets (BHS; 400 μm thickness, 16 mm diameter), an established in vitro surrogate for human nails in transungual permeation studies, were soaked in excess in the three test products and the three reference products for 24 hours. Afterwards the BHS were taken from the soaking baths and remaining residues of the test and reference products were thoroughly removed. Then, BHS were transferred and placed onto freshly plated T. interdigitale culture plates. Following incubation at 28° C. for 5, 7, 11 or 14 days, zones of inhibition were recorded and measured for each of the test and reference products. Experimental details and results are depicted in Table 2 below:

TABLE 2

| | | Incubation | | | |
|---|---|---|---|---|---|
| No. | Formulation | at 28° C. | | Replicates | Results |
| 1 | DMSO (negative | 5 | days | 3 | no inhibition zone |
| | control) | | | | |
| 2 | terbinafine (positive | 5 | days | 3 | nearly complete inhibition |
| | control, 100 μg/ml in | | | | zone (zones of inhibition: |
| | DMSO) | | | | 5.6-5.8 cm) |
| 3 | F6H8 (vehicle control) | 5 | days | 3 | no inhibition zone |
| 4 | 1% efinaconazole in | 5 | days | 1 | complete inhibition zone |
| | F6H8/2-propanol (9:1) | | | | |
| 5 | 1% efinaconazole in | 7 | days | 3 | complete inhibition zone |
| | F6H8/2-propanol (9:1) | | | | |
| 6 | 1% efinaconazole in | 11 | days | 1 | complete inhibition zone |
| | F6H8/2-propanol (9:1) | | | | |
| 7 | 1% efinaconazole in | 14 | days | 3 | complete inhibition zone |
| | F6H8/2-propanol (9:1) | | | | |
| 8 | 0.1% efinaconazole in | 4 | days | 1 | nearly complete inhibition |
| | F6H8/2-propanol | | | | zone (zone of inhibition: |
| | (10:1) | | | | 5.9 cm) |
| 9 | 0.1% efinaconazole in | 11 | days | 1 | nearly complete inhibition |
| | F6H8/2-propanol | | | | zone (zone of inhibition: |
| | (10:1) | | | | 5.9 cm) |
| 10 | Jublia (10% | 5 | days | 1 | complete inhibition zone |
| | efinaconazole, aqueous) | | | | |
| 11 | Jublia (10% | 11 | days | 1 | complete inhibition zone |
| | efinaconazole, aqueous) | | | | |

### Results:

The positive control (2) (terbinafine dissolved at 100 μg/ml in DMSO) and negative (DMSO) control (1) showed the expected positive (inhibition zones: 5.6-5.8 cm) and negative outcomes (no inhibition zones). While the vehicle control (F6H8) (3) did not generate an inhibition zone, the efinaconazole formulations (4-11), including the non-aqueous (0.1% and 1%) and the 10% aqueous formulations did generate nearly complete to complete inhibition zones.

Interestingly, the non-aqueous 1% efinaconazole formulation (4-7) resulted in comparable inhibition of growth of T. interdigitale, as compared to the 10-times higher concentrated commercial aqueous 10% efinaconazole formulation (Jublia) (10-11). Unexpectedly, also the 100-times lower concentrated non-aqueous 0.1% efinaconazole formulation (8-9) did achieve nearly complete inhibition of growth (inhibition zone: 5.9 cm), not less than the positive control (terbinafine) (2).

Without being bound by theory, it is believed that efinaconazole formulated in SFAs (i.e. F6H8) unfolds the full antifungal potential of efinaconazole, which may be hampered by the presence of one or more excipients in the commercial Jublia formulation. The commercial efinaconazole formulation (10-11), besides the antioxidant butylated hydroxytoluene (BHT), contains as excipients alcohol, anhydrous citric acid, C12-15alkyl lactate, cyclomethicone, diisopropyl adipate, disodium edetate, purified water. Instead, the non-aqueous efinaconazole formulations (4-9) is formulated without antioxidants, containing only 2 components, namely the semifluorinated alkane F6H8 and 2-propanol.

### Example 4: In Vitro Killing and Regrowth

Two test products containing 2% and 10% efinaconazole were tested in vitro for their capabilities to kill dermatophyte (T. interdigitale) in a previously infected Bovine Hoof Sheet and prevent regrowth of the dermatophyte:
(a) 2.0% efinaconazole, dissolved in a mixture of F6H8 and 2-propanol (9:1 v/v %), non-aqueous (14),
(b) 10% efinaconazole, aqueous (commercial product Jublia (Bausch Health, Bridgewater, USA)) (15)

As reference products, water was utilized as negative control and 7.5% povidone-iodine (Braunol, B. Braun Melsungen AG, Melsungen, Germany) as positive control (13). The non-aqueous 2% efinaconazole formulation (14) did not contain any antioxidant, presented as colourless solution and did not show any discoloration during performance of the testing.

Bovine Hoof Sheets (BHS; 400 μm thickness, 16 mm diameter), an established in vitro surrogate for human nails in transungual permeation studies, were first infected with the dermatophyte T. interdigitale. Afterwards the infected BHS were incubated for 1 hour in the 2 test products and the reference products. Then, the BHS were removed from the incubation. Subsequent thorough washing of the BHS ensured that no remains of the test and reference products adhered to the surface of the BHS. After washing the BHS were transferred to fresh culture plates, incubated for 4 days at 28° C. and a potential regrowth of T. interdigitale from the BHS was recorded. Experimental details and results are depicted in Table 3 below.

TABLE 3

| | | Incubation | | |
|---|---|---|---|---|
| No. | Formulation | at 28° C. | Replicates | Result |
| 12 | water | 4 days | 3 | full lateral |
| | (negative control) | | | regrowth |
| 13 | 7.5% Povidone iodine | 4 days | 3 | minor lateral |
| | (positive control) | | | regrowth |
| 14 | 2% efinaconazole in | 4 days | 3 | no regrowth |
| | F6H8/2-propanol (9:1) | | | |
| 15 | Jublia (10% | 4 days | 3 | no regrowth |
| | efinaconazole) | | | |

### Results:

The negative control (water) showed the expected uniform lateral regrowth of T. interdigitale on the culture plates, while the positive control (7.5% povidone-iodine) killed the dermatophytes in the BHS only partially, leading to minor lateral regrowth on the culture plates.

Instead, both efinaconazole formulations, including the non-aqueous SFA-based (2%) and the aqueous (10%) formulations did effectively penetrate into the BHS and completely kill the dermatophytes inside the BHS and therefore prevented regrowth of T. interdigital from the BHS on the culture plates. This represents an important characteristic of a long-term effective treatment of onychomycosis and/or tinea pedis.

Interestingly, the non-aqueous 2% efinaconazole formulation (14) resulted in comparable prevention of regrowth of T. interdigitale from an infected BHS, as compared to the 5-times higher concentrated commercial Jublia (10%) formulation (15). Without being bound by theory, it is believed that the 5-times lower concentrated efinaconazole formulation in SFAs (i.e. F6H8) (14) unfolds its improved antifungal potential due to improved penetration capabilities of the vehicle and further due to the absence of one or more excipients that may hamper the penetration and/or effectivity of efinaconazole in the commercial aqueous Jublia formulation (15).

### Example 5: Discoloration & Stability

Certain aqueous efinaconazole formulations are known to discolour over time from colourless to pale yellow to deep red or brown over time, which can discourage patients from use. Additionally, the clinical effectiveness of the active ingredient could be diminished by oxidative change. Thus, the non-aqueous SFA-based (i.e. F6H8) formulations (16-17) was evaluated with regard to visual appearance and discoloration, including stress conditions (60° C.). Experimental details and results are depicted in Table 4 below:

TABLE 4

| | | | Absorbance (400 nm) | | | |
|---|---|---|---|---|---|---|
| | | | | 2 weeks, | 4 weeks, | 6 weeks, |
| No. | Formulation | Appearance | initial | 60° C. | 60° C. | 60° C. |
| 16 | 1% (w/w) efinaconazole | colourless | 0.010 | 0.018 | 0.010 | nd |
| | in F6H8/2-Propanol | | | | | |
| 17 | 0.2% (w/w) | colourless | 0.003 | 0.009 | 0.003 | nd |
| | efinaconazole in | | | | | |
| | F6H8/2-Propanol | | | | | |

### Results:

The less complex, antioxidant-free, non-aqueous efinaconazole formulations in SFAs (16-17) are colourless and present as highly stable formulations. Even after storage of the SFA-based efinaconazole formulations (16-17) under stress conditions (60° C.) no change in appearance (colour) or absorbance was evident.

### Example 6: Drying after Topical Application to the Nail

From a user-perspective it is preferrable that any antifungal formulation applied to the nail penetrates and dries fast, leading to higher patient compliance. Two test products containing 1% and 10% efinaconazole were tested in vitro for their rate of penetration into Bovine Hoof Sheets (BHS).
(a) 1.0% efinaconazole, dissolved in a mixture of F6H8 and 2-propanol (9:1 v/v %), non-aqueous (18)
(b) 10% efinaconazole, aqueous (commercial product Jublia (Bausch Health, Bridgewater, USA) (19)

Bovine Hoof Sheets (BHS; 400 μm thickness, 16 mm diameter), an established in vitro surrogate for human nails in transungual permeation studies were place on a white paper. Then, 3 μl of each test products were applied onto the surface of the Bovine Hoof Sheets. The amount was limited to 3 μl to avoid spreading over the surface of the sheets. The rate of penetration, meaning the time for complete penetration of the test products into the nail material of the BHS was determined visually. Herein, the reflection of light onto the BHS was conveniently utilized to determine the duration/time till the test products completely penetrated into the BHS or evaporated. Experimental details and results are depicted in Table 5 below:

TABLE 5

| | | Appearance of the BHS-surface | | | |
|---|---|---|---|---|---|
| | | time | | | |
| No. | Formulation | 0 min | 10 min | 20 min | 1 h |
| 18 | 1% (w/w) | Wet, full light | Almost dry, | Dry, no | Dry, no |
| | efinaconazole in | reflection | light | reflection | reflection |
| | F6H8/2-propanol | | shimmering of | | |
| | | | the surface | | |
| 19 | Jublia (10% | Wet, full light | Wet, full light | Partly dry, | Shimmering of |
| | efinaconazole), | reflection | reflection | strong | the surface |
| | aqueous | | | shimmering of | |
| | | | | the surface | |

### Results:

The non-aqueous 1% efinaconazole formulation (18) cleared away quickly from the surface of the BHS. After 10 min the formulation only minor quantities were present, which disappeared completely after 20 min. Interestingly, no remains of the drug compound, nor the vehicle were recognizable anymore on the nail surface. Thus, it was concluded that the non-aqueous efinaconazole formulation (18) completely penetrated into the nail material. Taking its high boiling point of 223° C. into account, is believed that the F6H8 does not simply evaporate away but provides for effective penetration of efinaconazole into the nail material.

In comparison, the commercial aqueous efinaconazole formulation (19) appeared to be present on the BHS for substantial longer time. It was observed that a glossy film has formed on the BHS, which was visible even longer than one hour, which was evident as a shimmering on the surface. This film feels dry to the touch. Therefore, it was concluded that at least a part of the commercial aqueous efinaconazole formulation (19) does not penetrate well into the nail but dries and remains as a glossy film on the nail surface, possibly embedding some of the efinaconazole active ingredient.

In summary, it can be concluded that the non-aqueous 1% efinaconazole formulation (18) did show a highly user-friendly behaviour, possibly leading to a higher patient compliance. Patient compliance is especially important, as topical antifungal treatment requires patients to apply the treatment over a period of at least 9-12 months to be effective.

### Example 7

### Preparation of a Solution of Terbinafine in Semifluorinated Alkanes

20 mg terbinafine (free base; purity >98%) is weighed in a 5-ml glass vial; 2 ml F6H8 (Novaliq, purity >99%) is added into the vial by using a pipette, closed with aluminium crimp cap and shaken overnight at 280 mpm, at RT. The resulting 10 mg/ml composition of terbinafine in F6H8 presents as clear solution.

Following the aforementioned procedure the following compositions were prepared (Table 6):

TABLE 6

| Terbinafine | semifluorinated | | |
|---|---|---|---|
| concentration | alkane | Cosolvent(s) | Appearance |
| 1 mg/mL | F6H8 | none | clear solution |
| 5 mg/mL | F6H8 | none | clear solution |
| 10 mg/mL | F6H8 | none | clear solution |
| 15 mg/mL | F6H8 | none | clear solution |
| 20 mg/mL | F6H8 | ethanol | clear solution |
| | (90 vol %) | (10 vol %) | |
| 20 mg/ml | F6H8 | 2-propanol | clear solution |
| | (90 vol %) | (10 vol %) | |
| 1 mg/mL | F4H5 | none | clear solution |
| 5 mg/mL | F4H5 | none | clear solution |
| 10 mg/mL | F4H5 | ethanol | clear solution |
| | | (10 vol %) | |
| 10 mg/ml | F6H8 | 2-propanol | clear solution |
| | (90 vol %) | (10 vol %) | |

### Example 8: In Vitro Nail Penetration/Inhibition Model

As described in Example 3, various test products containing 0.1 to 10% efinaconazole were tested in vitro for their capabilities to inhibit growth of a representative dermatophyte (T. interdigitale), in particular to evaluate onset of action:
a) 0.1% to 10% efinaconazole, incubation for 24 hours

In a set of experiments the influence of different concentration of efinaconazole on inhibition of growth at longer incubation times (24 hours) was investigated. Experimental details and results are depicted in Table 7 below:

TABLE 7

| | Concentration | Inhibition |
|---|---|---|
| Formulation | (w/v) | zone/cm |
| Negative control: F6H8 | 0.0% | 0.0 |
| efinaconazole in F6H8/2-propanol (9:1) | 0.1% | 1.7 |
| efinaconazole in F6H8/2-propanol (9:1) | 0.5% | 4.4 |
| efinaconazole in F6H8/2-propanol (9:1) | 1.0% | 5.8 |
| Jublia (diluted with ethanol) | 1.0% | 6.5 |
| efinaconazole in F6H8/2-propanol (9:1) | 2.0% | 6.2 |
| Jublia (diluted with ethanol) | 2.0% | 5.9 |
| Jublia (commercial, aqueous) | 10.0% | 6.4 |

### Results:

When considering 24-hour incubation, the non-aqueous efinaconazole formulations resulted in effective inhibition of growth of T. interdigitale at concentrations of about 0.5 to 2% efinaconazole. The 1% and 2% non-aqueous efinaconazole formulations did achieve comparable inhibition of growth as compared to the aqueous diluted Jublia formulations, including the much higher concentrated commercial Jublia formulation.
b) 1.0% to 10% efinaconazole, incubation for 30 minutes

In comparison to a), the influence of different concentration of efinaconazole on inhibition of growth at short incubation times (30 min) was investigated. 10 Experimental details and results are depicted in Table 8 below:

TABLE 8

| | | Inhibition |
|---|---|---|
| Formulation | Concentration | zone/cm |
| Negative control: F6H8 | 0.0% | |
| efinaconazole in F6H8/2-propanol (9:1) | 1.0% | 6.9 |
| Jublia (diluted with ethanol) | 1.0% | 4.0 |
| efinaconazole in F6H8/2-propanol (9:1) | 2.0% | 5.9 |
| Jublia (diluted with ethanol) | 2.0% | 4.0 |
| Jublia (10% efinaconazole, aqueous) | 10.0% | 6.1 |

### Results:

When considering a rather short incubation time of 30 minutes, the 1% and 2% non-aqueous efinaconazole formulations clearly outperformed the 1% and 2% aqueous diluted Jublia formulations, and the much higher concentrated commercial Jublia formulation. Without being bound by theory, it shows that the formulation of efinaconazole formulated in semifluorinated alkanes, penetrate very quickly and effectively into the nail material, more quickly as compared to the aqueous efinaconazole formulations. Thus, the 1 1% and 2% non-aqueous efinaconazole formulations in semifluorinated alkanes present as highly effective formulation, considering both short (30 min) and long (24 hours, see above) incubation times.
c) 2% efinaconazole, incubation for 5 min to 24 hours

In a set of experiments a head-to-head comparison of 2% non-aqueous vs. aqueous efinaconazole formulations was investigated, covering a time-course series of incubation times from 5 min to 24 hours. Experimental details and results are depicted in Table 9 below:

TABLE 9

| | | Inhibition zone/cm | | | | | |
|---|---|---|---|---|---|---|---|
| | Concen- | 5 | 15 | 30 | 60 | 4 | 24 |
| Formulation | tration | min | min | min | min | hr | hr |
| efinaconazole in | 2.0% | 5.5 | 5.5 | 5.9 | 6.1 | 5.6 | 6.2 |
| F6H8/2-propanol (9:1) | | | | | | | |
| Jublia (diluted with | 2.0% | 2.3 | 3.0 | 4.0 | 4.5 | 5.4 | 5.9 |
| ethanol) | | | | | | | |

### Results:

The quick onset of action of non-aqueous efinaconazole formulations in semifluorinated alkanes was demonstrated in a head-to-head comparison of non-aqueous vs aqueous 2% efinaconazole formulations. It was confirmed that the 2% non-aqueous efinaconazole formulation was highly superior at shorter incubation times (5 min, 15 min, 30 min, 60 min) and even slightly better at longer incubation times (4 hr, 24 hr). Thus, the non-aqueous efinaconazole formulations in semifluorinated alkanes, are characterized by a very quick onset of action and persistent performance, which is clear advantage when compared to the aqueous efinaconazole formulations.

1. A water-free liquid composition comprising efinaconazole dissolved in a vehicle comprising a semifluorinated alkane.

2. The composition according to any of the preceding claims, wherein the semifluorinated alkane is selected from F4H5, F4H6, F4H8, F4H10, F6H6, F6H8, F6H10.

3. The composition according to any of the preceding claims, wherein the composition does not comprise a further active ingredient, optionally the composition does not comprise a further antifungal agent, preferably the composition does not comprise tavaborole.

4. The composition according to any preceding claim, wherein the composition comprises a cosolvent is selected from ethanol, 2-propanol, DMF (N,N-dimethylformamide), DMSO (dimethyl sulfoxide), DMAC (N,N-dimethylacetamide), NMP (1-Methyl-2-pyrrolidon).

5. The composition according to any preceding claim, wherein efinaconazole is present at a concentration of up to 2% (w/v).

6. The composition according to any preceding claim, wherein the composition comprises efinaconazole and less than about 0.1% (w/v) of efinaconazole-N-oxid.

7. The composition according to any preceding claim, being free of one or more further components selected from
i. ethanol
ii. penetration enhancer selected from transcutol and isopropyl myristate
iii. chelating agent selected from EDTA, DTPA
iv. wetting agent or surface tension lowering agent selected from cyclomethicone)
v. pH adjusting agent
vi. moisturizer selected from C12-15alkyl lactate)
vii. emollient selected from diisopropyl adipate)
viii. water
ix. preservative
x. surfactant
xi. propylene glycol, ethyl acetate or amyl acetate.

8. The composition according to any preceding claim, comprising or consisting of efinaconazole dissolved in vehicle comprising or consisting of 1-perfluorohexyloctane, and optionally 2-propanol.

9. The composition according to any preceding claim, comprising or consisting of about 0.5 to 2% (w/v) efinaconazole dissolved in 98 to 99.5% (v/v) of a vehicle comprising or consisting of 1-perfluroohexyloctane and 2-propanol.

10. The composition according to any preceding claim, consisting of up 2.0% (w/v) efinaconazole, up to 90% (w/v) of 1-perfluorohexyloctane and less than or equal to 10% (w/v) of 2-propanol, preferably consisting of up 2.0% (w/v) efinaconazole, up to 90% (v/v) of 1-perfluorohexyloctane and less than or equal to 10% (v/v) of 2-propanol.

11. The composition according to any preceding claim, consisting of 1% (w/v) or of 2.0% (w/v) efinaconazole, up to 90% (v/v) of 1-perfluorohexyloctane and less than or equal to 10% (v/v) of 2-propanol.

12. The composition according to any preceding claim, for use as medicine.

13. The composition for use according to claim 12, for use in the treatment of a fungal infection of the skin, preferably for treatment of a fungal infection of the nail (onychomycosis).

14. The composition for use according to claims 12 to 13, wherein the composition is administered by use of a sponge applicator, dropper, pen or a pipette to the fingernail and/or the toenail.

15. The composition for use according to claims 12 to 14, wherein the composition is topically administered by dropping one single drop to the surface of the nail (fingernail or toenail) of a subject,
i. without contacting the nail, the folds, the bed, the hyponychium and/or the undersurface of the plate of a toenail and/or a fingernail and/or
ii. without manually distributing the composition to one or more remote regions of the toenail and/or a fingernail selected from the folds, the bed, the hyponychium and/or the undersurface of the plate.

16. The composition for use according to claim 12, for use in the treatment of a fungal infection of the eye, preferably for treatment of a fungal infection of the cornea.

17. A kit comprising the composition as defined in any of the claims 1 to 11, and a container for holding the composition, wherein the contained is adapted for topical administration of the composition to the nail, the folds, the bed, the hyponychium and/or the undersurface of the plate of a toenail and/or a fingernail of a subject.
